(19) United States
(12) Patent Application Publication
(10) Pub. No.: US 2022/0331572 A1
(43) Pub. Date: Oct. 20, 2022

(54) MICRONEEDLE, MICRONEEDLE ARRAY, AND PRODUCTION METHOD OF MICRONEEDLE

(71) Applicant: TOHOKU UNIVERSITY, Sendai-shi (JP)

(72) Inventors: Matsuhiko NISHIZAWA, Sendai-shi (JP); Yuina ABE, Sendai-shi (JP); Ryohei TAKIZAWA, Sendai-shi (JP)

(73) Assignee: TOHOKU UNIVERSITY, Sendai-shi (JP)

(21) Appl. No.: 17/691,625

(22) Filed: Mar. 10, 2022

(30) Foreign Application Priority Data
Apr. 19, 2021 (JP) 2021-070360

(51) Int. Cl. A61M 37/00

(52) U.S. Cl. CPC A61M 37/0015; A61M 2037/0053; A61M 2205/0238; A61M 2206/10; A61M 2037/0023

(57) ABSTRACT

A porous microneedle capable of accessing a target portion on a surface of the skin or under the skin in a pinpoint manner, a microneedle array of the same, and a production method of the microneedle. A part of a surface of a porous needle main body is coated with a coating member. The needle main body may include a flow path inside formed to extend in a net shape. The needle main body may be formed of a porous body, and may include a flow path formed from void parts of the porous body.

### FIELD OF THE INVENTION

The present invention relates to a microneedle, a microneedle array, and a production method of microneedle.

### DESCRIPTION OF RELATED ART

A microneedle, which refers to a micro-sized needle having a length of approx. several hundreds of micrometers, is capable of accessing the inside of the skin with low invasion while suppressing pain to the minimum. Therefore, such a microneedle has been applied as a tool for transdermal drug delivery or intradermal substance sensing by breaking the barrier of the surface layer of the skin (refer to, for example, Non-Patent Literature 1 or 2).

As a basic microneedle, a hollow microneedle 51 shown in FIG. 9A has been provided (refer to, for example, Non-Patent Literature 3). The hollow microneedle 51 has the structure in which a conventional needle having a through hole inside the needle is ultra-miniaturized, and allows the opening of the hole to access a target portion under the skin in a pinpoint manner. However, the opening is hardly arranged at the top of the needle by considering influences given on the sharpness and strength of the needle, and the top opening if arranged lowers the sharpness of the needle. In addition, the formation of such a hollow shape requires a relatively complex machining technique, and further a fragment of living body tissue may enter and block the hole during use.

There is a porous microneedle 52 shown in FIG. 9B developed as a microneedle free from the problems above (refer to, for example, Patent Literature 1 or Non-Patent Literature 4). The porous microneedle 52 may include a needle main body that is formed of a porous body and includes interconnected voids inside functioning as a flow path, so that the entire surface of the needle main body including its top is connected to the flow path. Alternatively, the porous microneedle 52 may include the needle main body that is formed of hydrogel having the structure of high polymer network with high definition or of xerogel obtained by drying the hydrogel, and the network may function as a flow path, and thereby the entire surface of the needle main body is connected to the flow path. A production method such as molding is easily applicable to the production of the porous microneedle 52 because of its simpler shape compared to the hollow microneedle 51, and the porous microneedle 52 has low resistance in liquid delivery (allows liquid to flow smoothly). Accordingly, such a porous microneedle is expected to be applied as a tool for transdermal drug delivery or for diagnosis (refer to, for example, Patent Literature 2, or Non-Patent Literature 5 or 6).

Moreover, along with the advancement of fine processing technology, a microneedle array in which a plurality of microneedles are arranged side by side on a single substrate has been developed (refer to, for example, Patent Literature 3 or 4).

### CITATION LIST

### Non Patent Literature

Non-Patent Literature 1: Liu, G-S. et al., “Microneedles for transdermal diagnostics: Recent advances and new horizons”, Biomaterials, 2020, 232, 119740
Non-Patent Literature 2: Chang, H., Zheng, M., Chew, S. W. T. and Xu, C., “Advances in the Formulations of Microneedles for Manifold Biomedical Applications”, Adv. Mater. Technol., 2020, 5, p.1-19
Non-Patent Literature 3: Lhernould, M. S., “Optimizing hollow microneedles arrays aimed at transdermal drug delivery”, Microsyst. Technol., 2013, 19, p.1-8
Non-Patent Literature 4: Liu, L., Kai, H., Nagamine, K., Ogawa, Y. and Nishizawa, M., “Porous polymer microneedles with interconnecting microchannels for rapid fluid transport”, RSC Adv., 2016, 6, p.48630-48635
Non-Patent Literature 5: Nagamine, K., Kubota, J., Kai, H., Ono, Y. and Nishizawa, M., “An array of porous microneedles for transdermal monitoring of intercellular swelling”, Biomed. Microdevices, 2017, 19, p.1-6
Non-Patent Literature 6: Kusama, S. et al., “Transdermal electroosmotic flow generated by a porous microneedle array patch”, Nat. Commun., 2021, doi : 10. 1038/s41467-021-20948-4 .

### Patent Literature

Patent Literature 1: WO 2016/195119
Patent Literature 2: WO 2017/183737
Patent Literature 3: WO 2020/179850
Patent Literature 4: JP 2020-141765 A

### SUMMARY OF THE INVENTION

The porous microneedles described in Patent Literatures 1, 2, and Non-Patent Literatures 4 to 6 have low resistance in drug delivery and allow a liquid drug to flow smoothly, and are thus suitably applicable as a tool for drug delivery or for diagnosis. However, the needle main bodies have the openings of the flow paths on the entire surfaces, and thus the needle main bodies are not capable of accessing a target portion on the surface of the skin or under the skin in a pinpoint manner. Therefore, in the use for drug delivery, there is a risk of causing a loss of drug.

The present invention has been made focusing on those problems. It is an object of the present invention to provide a porous microneedle capable of accessing a target portion on the surface of the skin or under the skin in a pinpoint manner, a microneedle array of the same, and a production method of the microneedle.

To attain the above object, the microneedle according to the present invention is characterized in that a part of the surface of a porous needle main body thereof is coated with a coating member.

The microneedle according to the present invention has a porous needle main body with a part of the surface thereof other than a desired part coated with a coating member, and is capable of accessing a target portion on the surface of the skin or under the skin in a pinpoint manner by using openings of the desired part. Accordingly, in an example, in the use for drug delivery under the skin by use of the flow path formed from opened voids, the microneedle enables efficient drug delivery to a target portion, thereby enabling to provide more efficient drug administration and to reduce drug loss. In another example, in the use for sensing of intradermal information as a tool for diagnosis, the microneedle is capable of efficiently obtaining the information at a desired position. The skin is configured with various tissues, and the depth of the boundary between the epidermal tissue corresponding to the outermost skin and the dermal tissue formed under the epidermal tissue is approx. 0.1 mm under the surface of the skin. For this reason, for example, the microneedle with an opened part in the length covering the both tissues is capable of collecting interstitial fluid from a target portion or measuring potential for use as an index of a skin health condition.

In the microneedle according to the present invention, the needle main body may include a flow path inside formed to extend in a net shape. The needle main body may be formed of a porous body, and may include a flow path formed from void parts of the porous body. In these cases, the needle main body may be configured of hydrogel having the structure of high polymer network with high definition, xerogel obtained by drying the hydrogel, hydrogel material, resin, oxide, metal, or biodegradable material.

In the microneedle according to the present invention, the needle main body may preferably have, on the surface thereof, a plurality of openings communicating with the flow path, and the coating member may be preferably disposed so as to coat some openings out of the plurality of openings. In the microneedle, a plurality of the flow paths may be preferably formed, and each of the openings may preferably communicate with at least one of the flow paths. In these cases, the microneedle is capable of accessing a target portion on the surface of the skin or under the skin by use of openings other than the openings coated with the coating member.

In the microneedle according to the present invention, the coating member may be disposed so as to coat any part of the surface of the needle main body. In an example, the coating member may coat the part of the surface other than the tip part of the needle main body, so that the tip part of the needle main body is opened. In another example, the coating member may coat only the surface of the flange part arranged around the rear end part of the needle main body, so that the area from the rear end part to the tip part of the needle main body is opened. In further another example, the coating member may coat the rear end part and the tip part of the needle main body, so that the middle part of the needle main body is opened. In the microneedle according to the present invention with any part coated with a coating member, the sharpness of the tip part of the needle main body is prevented from decreasing.

The coating member may be made of any material as long as the material blocks the openings of the surface of the needle main body, and may be a protective film made of resist material, as an example. It is preferable that the material of the coating member is properly selected according to the use. In the use for electrical measurement, the coating member may be preferably made of an insulating material that is electrically nonconductive, for example, parylene. The coating member is not limited to a solid, but may be liquid or gel.

The microneedle array according to the present invention has a plurality of the microneedles according to the present invention, and the microneedles are arranged side by side.

The microneedle array according to the present invention, in which the plurality of microneedles according to the present invention are arranged side by side, is capable of, for example, efficiently measuring swelling by measuring resistance of the epithelium of the skin, or efficiently administering a drug in a line manner or a plane manner. In the microneedle array according to the present invention, the microneedles may be arranged in any configuration. In an example, the microneedles may be arranged side by side on a surface of a hard substrate or a flexible substrate. In another example, the microneedles may be arranged to be connected mutually.

The microneedle and the microneedle array according to the present invention may be produced in any method. The production method of microneedle according to the present invention for producing the microneedle and the microneedle array according to the present invention may include, for example, a needle forming process of producing the needle main body with the flow path blocked with a soluble material dissolvable in a predetermined solution, a coating process of first masking a part of the surface of the needle main body with a protective member, and subsequently applying the coating member on the surface of the needle main body, and a flow path forming process of first removing the protective member, and subsequently dissolving the soluble material in the predetermined solution.

In the use of the protective member, the needle forming process may be performed by any method as long as the method enables to produce the needle main body with the flow path blocked with a soluble material, for example, a well-known method disclosed in any of Non-Patent Literatures 4 to 6 and Patent Literatures 1 to 4. In the use of such a well-known method, the needle forming process may include the step of producing the porous microneedle in such a well-known method, and the step of blocking the flow path with a soluble material, or may be performed by use of the needle main body with the flow path blocked with a soluble material, which is obtained during the production of the porous microneedle by a well-known method. In the needle forming process, the entire flow path may be blocked with a soluble material, or alternatively only the openings of the flow path on the surface of the needle main body may be blocked.

In the use of the protective member, the soluble material may be made of any material as long as the material is different from the material of the needle main body. The predetermined solution for dissolving the soluble material is preferably the solution that dissolves only the soluble material but does not dissolve the needle main body, and may be made of any material of such characteristics.

In another example of the production method of microneedle according to the present invention, the production method of microneedle may include the steps of, without using the soluble material, preparing the needle main body with the flow path thereof not blocked, that is, the needle main body with the flow path formed, masking a part of the surface of the needle main body with a protective member, coating the surface of the needle main body with the coating member, and subsequently removing the protective member. In further another example, the method may include the steps of, without using the protective member or the soluble material, preparing the needle main body with the flow path thereof not blocked, that is, the needle main body with the flow path formed, and coating a part of the surface of the needle main body with the coating member so as not to block the openings of other part. In further another example, the method may include the step of producing the needle main body with all the openings of the flow path thereof blocked with the soluble coating member, and before use or during use, only the part of the soluble coating member blocking the openings of a desired position may be dissolved.

The present invention provides a porous microneedle capable of accessing a target portion on the surface of the skin or under the skin in a pinpoint manner, a microneedle array of the same, and a production method of the microneedle.

### BRIEF DESCRIPTION OF THE DRAWINGS

FIG. 1A is a sectional view of a microneedle in an embodiment of the present invention, having a structure in which an area from a rear end part to a tip part of a needle main body of the microneedle is opened; FIG. 1B is a sectional view of the microneedle having a structure in which only the tip part of the needle main body is opened; FIG. 1C is a sectional view of the microneedle having a structure in which the tip part of the needle main body is opened widely; and FIG. 1D is a sectional view of the microneedle having a structure in which a middle part of the needle main body is opened.

FIGS. 2A to 2D are sectional views of a needle forming process of steps, in the production method of microneedle in the embodiment of the present invention.

FIGS. 3A and 3B are sectional views of a coating process of steps and FIGS. 3C and 3D are sectional views of a flow path forming process of steps, in the production method of microneedle in the embodiment of the present invention.

FIG. 4A is a perspective view of a method of masking a microneedle array in which three or more microneedles are integrated and FIG. 4B is a perspective view of a method of masking microneedle arrays each in which only one microneedle is arranged on one substrate, in the coating process in the production method of microneedle in the embodiment of the present invention.

FIG. 5A is a microscopic photograph taken by a scanning electron microscopy (SEM) indicating a position of line scanning by EDX in the vicinity of the tip part of the needle main body of the microneedle in the embodiment of the present invention; and FIG. 5B is a graph of C1 signal intensity in the results of the line scanning.

FIG. 6A is a sectional view of the microneedle in the embodiment of the present invention, in filling with coloring matter, and FIG. 6B is a sectional view of the microneedle with the coloring matter diffused, in a method of a diffusion test of coloring matter; and FIG. 6C is a microscopic photograph of an uncoated microneedle (a comparative example), and FIG. 6D is a microscopic photograph of a microneedle with a part coated other than the tip part, in the results of the diffusion test of coloring matter.

FIG. 7A is a sectional view of the microneedle in the embodiment of the present invention in filling with an electrolyte solution, and FIG. 7B is a sectional view of the microneedle in application of a DC current for measurement of resistance values, in a test method of measuring the DC resistance values; and FIG. 7C is a graph of the resistance values of the uncoated microneedle (the comparative example), the microneedle with a part coated other than the tip part, and a microneedle coated entirely (another comparative example), in the results of the measurement of the DC resistance values.

FIG. 8A is a sectional view of the microneedle in the embodiment of the present invention in a method of measuring epidermal potential; FIG. 8B is an enlarged sectional view of the inserted microneedle; and FIG. 8C is a graph of the epidermal potential of the microneedle with a part coated other than the tip part and the uncoated microneedle (the comparative example) in the results of the measurement of the epidermal potential, and the epidermal potential measured by a conventional method.

FIG. 9A is a sectional view of a conventional hollow microneedle; and FIG. 9B is a sectional view of a conventional porous microneedle.

### DETAILED DESCRIPTION OF THE INVENTION

Embodiments of the present invention will be described with reference to the drawings, hereinafter.

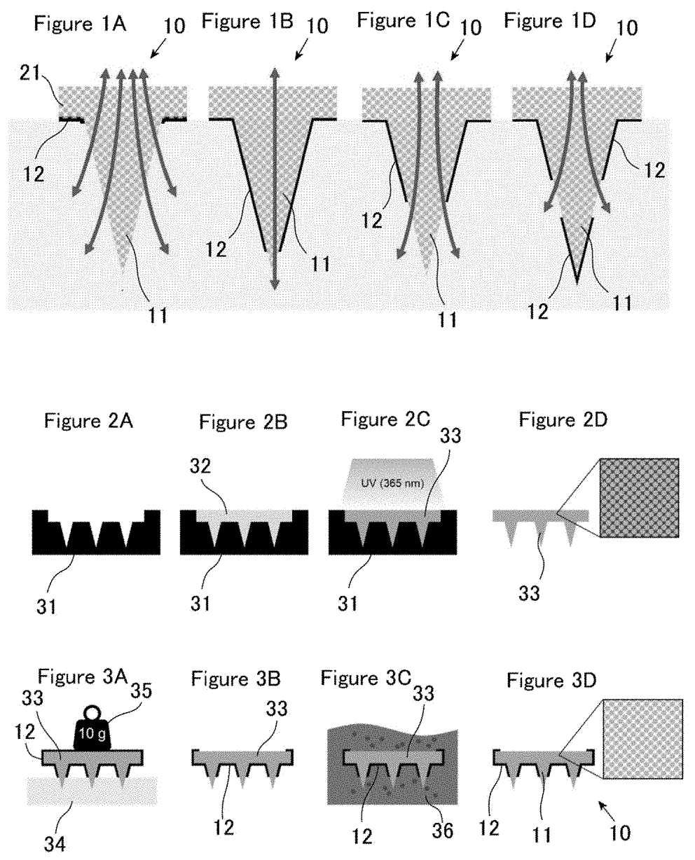

FIGS. 1 to FIGS. 8 show the microneedle, the microneedle array and the production method of microneedle according to the embodiment of the present invention.

As shown in FIG. 1A to FIG. 1D, a microneedle 10 in the embodiment of the present invention includes a needle main body 11 and a coating member 12. In the microneedle array in the embodiment of the present invention, a plurality of the microneedles 10 are arranged side by side.

The needle main body 11 is a porous member formed in a conical shape, and has a flange part 21 around the rear end part in the bottom side of the cone. The needle main body 11 has a flow path inside, and has a plurality of openings communicating with the flow path, on the surface. The flow path may be formed to extend in a net shape inside the needle main body 11, or may be formed from void parts of the needle main body 11 formed of a porous body. It is preferable that a plurality of the flow paths are formed, and each of the openings communicates with at least one of the flow paths. In an example, the needle main body 11 is configured of hydrogel having a structure of high polymer network with high definition, xerogel obtained by drying the hydrogel, hydrogel material, resin, oxide, metal, or biodegradable material.

The hydrogel material herein refers to the material that forms hydrogel by being dispersed in water (dispersion medium). Examples of the hydrogel material include natural polymers of agar, gelatin, agarose, xanthan gum, gellan gum, sclerotium gum, gum arabic, gum tragacanth, gum karaya, cellulose gum, tamarind gum, guar gum, locust bean gum, glucomannan, chitosan, carrageenan, quince seed, galactan, mannan, starch, dextrin, curdlan, casein, pectin, collagen, fibrin, peptide, chondroitin sulfate such as chondroitin sulfate sodium salt, hyaluronate such as hyaluronic acid (mucopolysaccharide) and sodium hyaluronate, alginate such as alginic acid, sodium alginate and calcium alginate, and their derivatives; cellulose derivatives of methylcellulose, hydroxymethyl cellulose, hydroxyethyl cellulose, hydroxypropyl cellulose, hydroxypropyl methylcellulose, carboxymethyl cellulose, and their salts; poly (meth) acrylic acids of polyacrylic acid, polymethacrylic acid, sodium polymethacrylate, and acrylic acid-alkyl methacrylic acid copolymer, and their salts; synthetic polymers of polyvinyl alcohol, polyhydroxyethyl methacrylate, polyacrylamide, poly (N-isopropyl acrylamide), polyvinylpyrrolidone, polystyrene sulfonate, polyethylene glycol, carboxyvinyl polymer, alkyl modified carboxyvinyl polymer, maleic anhydride copolymer, polyalkylene oxide resin, crosslinked product of poly (methyl vinyl ether-alt-maleic anhydride) and polyethylene glycol, polyethylene glycol crosslinked product, N-vinyl acetamide crosslinked product, acrylamide crosslinked product, and crosslinked product of starch-acrylate graft copolymer; silicone; interpenetrating network hydrogel and semi-interpenetrating network hydrogel; poly 2-hydroxyethyl methacrylic acid, and poly 2-acrylamide-2-methyl propane sulfonic acid; and mixtures of two or more materials among these. Preferable hydrogel materials among these materials, from the viewpoint of withstand load and bioaffinity, are collagen, and glucomannan; carboxymethyl cellulose, and sodium carboxymethyl cellulose; polyacrylic acid, and sodium polyacrylate; and interpenetrating network hydrogel, and semi-interpenetrating network hydrogel. A preferable hydrogel material, from the viewpoint of excellent mechanical strength and excellent biocompatibility, is a crosslinked product of poly (methyl vinyl ether-alt-maleic anhydride) and polyethylene glycol. A preferable hydrogel material, from the viewpoint of ensuring electrical neutrality in hydrogel, is crosslinked polyethylene glycol.

Examples of the resin include polycarbonate, acrylonitrile butadiene styrene (ABS) resin, phenolic resin, acrylic resin, methacrylic resin (such as, polyglycidyl methacrylate resin). Examples of the oxide include inorganic oxide and its derivative. Examples of the inorganic oxide herein include silicon oxide, tin oxide, zirconia oxide, titanium oxide, niobium oxide, tantalum oxide, aluminum oxide, tungsten oxide, hafnium oxide, and zinc oxide. Examples of the metal include nickel, iron, and these alloy. Examples of the biodegradable material include poly lactic-co-glycolic acid (PLGA) and mixed material mainly made of PLGA, β-tricalcium phosphate, calcium carbonate, polycaprolactone, polydioxanone, hydroxyapatite, polyethylene glycol, and magnesium alloy. The needle main body 11 may be made of combination of two or more of the substances indicated above.

The coating member 12 is disposed so as to coat a part of the surface of the needle main body 11. The coating member 12 is disposed so as to coat some openings out of the plurality of openings of the surface of the needle main body 11. The coating member 12 may coat any part of the surface of the needle main body 11. In an example, as shown in FIG. 1A, in the microneedle 10, the coating member 12 may coat only the surface of the flange part 21 arranged around the rear end part of the needle main body 11, so that the area from the rear end part to the tip part of the needle main body 11 is opened. As shown in FIG. 1B, the coating member 12 may coat a part of the surface other than the tip part of the needle main body 11, so that only the tip part of the needle main body 11 is opened. As shown in FIG. 1C, the coating member 12 may coat a part of the surface other than a large area around the tip part of the needle main body 11, so that the tip part of the needle main body 11 is opened widely. As shown in FIG. 1D, the coating member 12 may coat the rear end part and the tip part of the needle main body 11, so that the middle part of the needle main body 11 is opened. The microneedles 10 described above allow fluid, an electrical signal or the like to flow through the flow paths as shown by the arrows in the FIGS.

The coating member 12 may be made of any material as long as the material blocks the openings of the surface of the needle main body 11, and may be a protective film made of resist material, as an example. It is preferable that the material of the coating member 12 is properly selected according to the use. In the use for electrical measurement, the coating member 12 may be preferably made of an insulating material that is electrically nonconductive, for example, parylene. The coating member 12 is not limited to a solid, but may be liquid or gel.

The microneedle 10 and the microneedle array according to the embodiment of the present invention are able to be produced by the production method of microneedle according to the embodiment of the present invention to be described below. In other words, the production method of microneedle according to the embodiment of the present invention includes the step of, in a needle forming process, first producing the needle main body 11 with its flow path blocked with a soluble material that is dissolvable in a predetermined solution. In the needle forming process, a well-known method of producing a microneedle may be used. In an example, after the production of a microneedle by a well-known method, the flow path may be blocked with a soluble material. Alternatively, the needle main body with its flow path blocked with a soluble material that is obtained in the process of producing the microneedle by a well-known method may be used. The soluble material herein may be made of any material as long as the material is different from the material of the needle main body 11. The predetermined solution that dissolves the soluble material may be any solution that does not dissolve the needle main body 11 but dissolves the soluble material.

After the needle forming process, a part of the surface of the needle main body 11 is masked with a protective member, and subsequently the coating member 12 is disposed on the surface of the needle main body 11 (coating process). Thereafter, the protective member is removed, and the soluble material is dissolved in a predetermined solution (flow path forming process). With these processes, the microneedle 10 and the microneedle array according to the embodiment of the present invention are able to be produced.

The microneedle 10 having the porous needle main body 11 with a part of the surface thereof other than a desired part coated with the coating member 12 is capable of accessing a target portion on the surface of the skin or under the skin in a pinpoint manner by using the openings of the desired part. Accordingly, for example, in the use for drug delivery under the skin, the microneedle 10 is capable of efficiently delivering a drug to a target portion, thereby enabling to provide more efficient drug administration and to reduce drug loss. In the use for sensing of intradermal information or the like as a tool for diagnosis, the microneedle 10 is capable of efficiently obtaining the information at a desired position. In an example, the microneedle 10 with an opened part in the length covering the both tissues of an epidermal tissue and a dermal tissue is capable of collecting interstitial fluid from a target portion or measuring potential for use as an index of a skin health condition.

In the microneedle 10 with any part coated with the coating member 12, the sharpness of the tip part of the needle main body 11 is prevented from decreasing. The microneedle array, in which the plurality of microneedles 10 are arranged side by side, is capable of efficiently measuring swelling by measuring resistance of the epithelium of the skin, or efficiently administering a drug in a line manner or a plane manner.

In another example of the production method of microneedle according to the embodiment of the present invention, the method may include the steps of, without using the soluble material, preparing the needle main body 11 with the flow path thereof not blocked, that is, the needle main body 11 with the flow path formed, masking a part of the surface of the needle main body 11 with a protective member, coating the surface of the needle main body 11 with the coating member 12, and subsequently removing the protective member. In further another example, the method may include the steps of, without using the protective member or the soluble material, preparing the needle main body 11 with the flow path thereof not blocked, that is, the needle main body 11 with the flow path formed, and coating a part of the surface of the needle main body 11 with the coating member 12 so as not to block the openings of other part. In further another example, the method may include the step of producing the needle main body 11 with all the openings of the flow path thereof blocked with the soluble coating member 12, and before use or during use, only the part of the soluble coating member 12 blocking the openings of a desired position may be dissolved.

### EXAMPLE 1

The microneedle 10 is produced by the production method of microneedle according to the embodiment of the present invention, and subjected to various types of evaluation experiments. The reagents, the materials and the instruments for use in the production and the experiments are as follows:

Glycidyl Methacrylate (GMA), made by FUJIFILM Wako Pure Chemical Corporation

Trimethylolpropane trimethacrylate (TRIM), made by Sigma Aldrich

Polyethylene glycol (PEG) 10 kDa, made by Sigma Aldrich

Diethylene glycol (DEG), made by Tokyo Chemical Industry Co., Ltd.

Irgacure 184, made by BASF SE

Parylene C, made by Daisan Kasei Co., Ltd.

Polydimethylsiloxane (PDMS), SILPOT 184, made by DuPont Toray Specialty Materials K.K.

Methanol, made by FUJIFILM Wako Pure Chemical Corporation

Rhodamine B, made by FUJIFILM Wako Pure Chemical Corporation

Gellan gum, made by FUJIFILM Wako Pure Chemical Corporation

Ethanol, made by FUJIFILM Wako Pure Chemical Corporation

Ringer solution, made by Otsuka Pharmaceutical Co., Ltd.

Acrylic board, made by Acrysunday Co., Ltd.

Cutting machine, made by Modia Systems Co., Ltd.

Scanning electron microscope (SEM) VE-9800, made by Keyence corporation

Energy dispersive X-ray spectroscopy system (SEM EDX), JSM-7400F, made by JEOL Ltd.

Electrochemical analyzer, ALS 7082E, made by BAS Inc.

The microneedle 10 is produced by any of the methods disclosed in Non-Patent Literatures 4 to 6. The microneedle array produced herein includes three of the microneedles 10 arranged side by side. First, the needle forming process shown in FIGS. 2A to 2D is performed. In other words, the acrylic board is cut by the cutting machine to produce a female mold having a microneedle shape. The female mold is transferred in two stages by use of the PDMS, and a female mold 31 made of the PDMS shown in FIG. 2A is produced.

Subsequently, a precursor solution for forming the porous needle main body 11 is prepared. The precursor solution is prepared by mixing photoinitiator in a solution A and a solution B. The solution A is prepared by mixing the PEG (4 g) of a soluble material and the DEG (20 mL) of a solvent at 60° C. The DEG, which has a molecular structure close to the PEG of a solute, is capable of preventing precipitation of the solute during the production process. Alternatively, 2-methoxyethanol may be used instead of the DEG The solution B is prepared by mixing the GMA (10 mL) of a monomer, and the TRIM (5.23 mL) and TEGDMA (15.7 mL) of cross-linking agents. The precursor solution is prepared by mixing the solution A (450 μL), the solution B (550 μL), and photoinitiator Irgacure 184 (1.8 mg) at 40° C. As shown in FIG. 2B, a precursor solution 32 prepared as above is poured in the female mold 31 made of the PDMS, and is subjected to degassing at 25° C. for 80 minutes under a reduced pressure of −0.096 MPa. The degassing process enables to prevent a defect in shape of the needle main body from occurring due to mixed bubbles.

As shown in FIG. 2C, the female mold 31 above after the degassing process is exposed to irradiation of ultraviolet rays with a wavelength of 365 nm at 25° C. for one hour, so that the monomer and the cross-linking agent are polymerized and solidified. Subsequently, as shown in FIG. 2D, a solidified molding 33 is taken out from the female mold 31. The resultant molding 33 has a needle shape, and the void parts of the needle main body 11 formed of a porous body are blocked with the PEG of porogen.

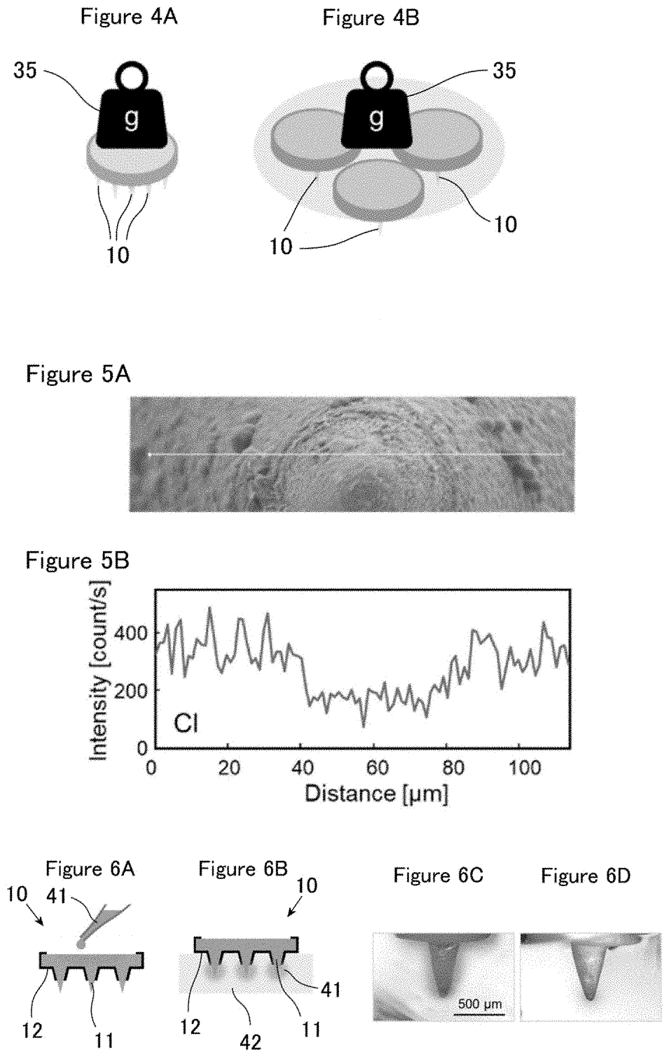

Subsequently, the coating process shown in FIG. 3A and FIG. 3B and the flow path forming process shown in FIG. 3C and FIG. 3D are performed. First, as shown in FIG. 3A, the molding 33 is placed on a PDMS flat plate 34 in such a manner that the tip part of the needle thereof faces downward, and a weight 35 is placed on the molding 33 for application of a constant load, so that the tip part of the needle is inserted into the PDMS flat plate 34 for masking. The PDMS flat plate 34 serves as a protective member. At this time, the microneedle array in which three of the microneedles are integrated is checked with respect to the change in masking state generated according to the amount of the load. This reveals that as the amount of the load is larger, the PDMS flat plate 34 sinks more deeply and the masking area is also larger. In the case of the microneedle array in which three or more of the microneedles 10 formed in the same length are integrated as shown in FIG. 4A, the microneedle array placed so as to face downward is capable of standing stably and independently. In the case of the microneedle 10 placed on one substrate on one-to-one basis, a plurality of the microneedles are placed and fixed on the same flat plate so as to face downward as shown in FIG. 4B, and thereby the microneedles 10 are placed stably. In one example shown in FIG. 3A, the weight 35 of 10 g is used so that stable and reliable masking is performed. It is noted that in order to open a wider area of the tip part by increasing the masking area of the tip part, the weight 35 that is heavier than 10 g (for example, the weight of 30 g or 50 g) may be used.

As shown in FIG. 3A, after the tip part of the needle of the molding 33 is inserted into the PDMS flat plate 34 for masking, the molding 33 is subjected to vapor deposition of the Parylene C by use of a parylene deposition apparatus so that the deposition thickness of 2 μm is formed. This forms the coating member 12. After the deposition, as shown in FIG. 3B, the coating member 12 on the substrate in the side opposite to the side of the needle of the molding 33 is removed by a cutting tool. As shown in FIG. 3C, the molding 33 with the coating member 12 formed is immersed overnight in a mixed solution 36 of distilled water and the methanol (1:1 in volume ratio), to elute the PEG In this way, the microneedle 10 as shown in FIG. 3D is produced, in which a part of the surface of the porous needle main body 11 is coated with the parylene coating member 12. The microneedles 10 are arranged integrally side by side on the surface of the substrate. The substrate serves as the flange parts 21 of the respective needle main bodies 11, and is made of the same material as the needle main bodies 11.

### Evaluation of Produced Microneedle

The produced microneedle 10 is subjected to line scanning in the vicinity of the tip part of the needle main body 11 by the EDX. FIGS. 5A and 5B show the results. The results shown in FIGS. 5A and 5B indicate that the intensity of the C1 signals derived from parylene is reduced in the vicinity of the tip part of the needle main body 11. With respect to the produced microneedle 10 and an uncoated microneedle (a comparative example), the porosities thereof calculated based on the amounts of water absorption by gravimetry are 40.7±1.3% (n=7) and 40.2±1.6% (n=8), respectively. The results indicate that the presence or absence of coating hardly affects the elusion of the PEG.

Subsequently, the produced microneedle is subjected to a diffusion test of coloring matter. As shown in FIG. 6A, with respect to the produced microneedle 10 and the uncoated microneedle (the comparative example), the coloring matter in an ethanol 41 with the rhodamine B dissolved therein (concentration of the rhodamine B: 10 mM) has been dropped on the respective microneedles from the substrate side opposite to the side of the needle main body 11 for 30 minutes, for sufficient absorption. Subsequently, as shown in FIG. 6B, the needle main bodies 11 are individually inserted into a gellan gum hydrogel 42 of 2 wt % to observe the diffusion of the coloring matter. FIG. 6C and FIG. 6D show the results. The results indicate that the uncoated microneedle diffuses the coloring matter from the whole of the needle as shown in FIG. 6C, while the produced microneedle 10 diffuses the coloring matter only from the tip part of the needle main body 11 as shown in FIG. 6D.

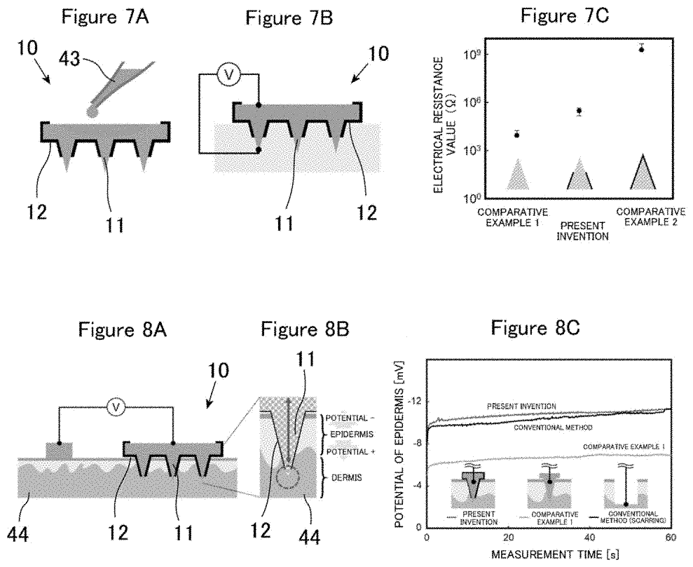

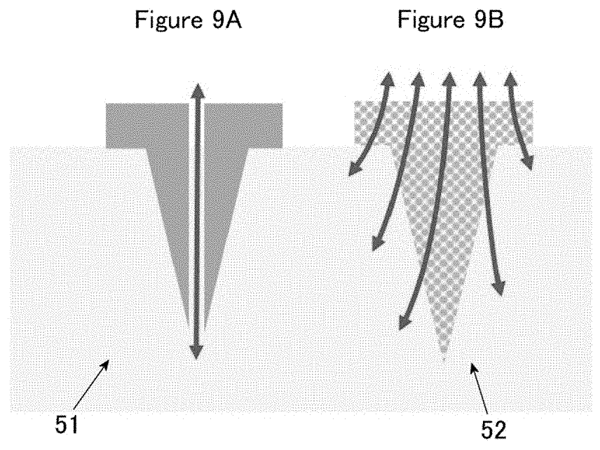

Subsequently, the produced microneedle is subjected to the test for measuring a DC resistance. As shown in FIG. 7A, with respect to the produced microneedle 10 (the present invention), the uncoated microneedle (a comparative example 1), and an entirely coated microneedle (a comparative example 2), the voids thereof are filled with an electrolyte solution (ringer solution) 43, and as shown in FIG. 7B, a DC current is applied to the microneedles by the electrochemical analyzer (in a constant current application mode) to calculate DC resistance values based on Ohm's law. FIG. 7C shows the results. As shown in FIG. 7C, as the coated area is larger, the resistance value is greater. The results indicate that insulating coating enables to limit current paths.

### Epidermal Potential Measuring Test

The epidermal potential measurement is performed by use of the microneedle 10 (the present invention), in which a small part of the tip part of the needle main body 11 is exposed and the other part is coated with the coating member 12. Since an epidermal potential is generated in the thickness direction of the epidermal tissue corresponding to the outermost skin, the uncoated microneedle that is exposed entirely is not capable of measuring the potential difference between the inside and the outside of the epidermis, due to short circuit. On the other hand, in the microneedle 10 allowing pinpoint connection at the tip part, the tip part of the needle main body 11 is connected to the dermis in a pinpoint manner under the epidermal tissue. Accordingly, the microneedle 10 is capable of measuring the potential difference between the inside and the outside of the epidermis.

As shown in FIG. 8A and FIG. 8B, the potential difference is measured between the surface of a pig skin sample 44 and the tip part of the needle main body 11 inserted to reach the dermis. For comparison, the measurement is also performed by use of the uncoated microneedle (the comparative example 1), and further performed by a conventional method (the method of making a scar reaching the dermis for conduction under the epidermal tissue). In order to ensure conduction via ions between the surface and the inside of the skin tissue, the microneedles and the hydrogel are made to contain an electrolyte solution (ringer solution) for use as salt bridge. The electrochemical analyzer (in a voltmeter mode) is used to measure the potential difference. FIG. 8C shows the measurement results.

As shown in FIG. 8C, the measured values of potential difference obtained by the conduction with the microneedle 10 through the tip part thereof and by the conduction performed by the conventional method are substantially the same, and are larger than the measured values obtained by the conduction with the uncoated microneedle. The results indicate that, in the conduction with the microneedle 10 through the tip part thereof, only the tip part of the needle main body 11 has the function of conduction, and is connected to the dermis in a pinpoint manner.

### REFERENCE SIGNS LIST

10: Microneedle
11: Needle main body
21: Flange part
12: Coating member
31: Female mold
32: Precursor solution
33: Molding
34: PDMS flat plate
35: Weight
36: Mixed solution
41: Ethanol with rhodamine B dissolved
42: gellan gum hydrogel of 2 wt %

43: Electrolyte solution (Ringer solution)
44: Pig skin sample

1. A microneedle having a porous needle main body, a part of a surface of the needle main body coated with a coating member.

2. The microneedle according to claim 1, wherein the needle main body includes a flow path inside formed to extend in a net shape.

3. The microneedle according to claim 1, wherein the needle main body is formed of a porous body, and includes a flow path formed from void parts of the porous body.

4. The microneedle according to claim 2, wherein
the needle main body has, on the surface, a plurality of openings communicating with the flow path, and
the coating member is disposed so as to coat some openings out of the plurality of openings.

5. The microneedle according to claim 4, the microneedle configured to, when being inserted into a skin, access a target portion on a surface of the skin or under the skin by use of openings other than the openings coated with the coating member.

6. The microneedle according to claim 4, wherein
a plurality of the flow paths are formed, and
each of the openings communicates with at least one of the flow paths.

7. The microneedle according to claim 1, wherein the coating member is disposed so as to coat a part of the surface other than a tip part of the needle main body.

8. The microneedle according to claim 1, wherein the coating member is made of an insulating material.

9. A microneedle array having a plurality of the microneedles according to claim 1, wherein
the microneedles are arranged side by side.

10. A production method of microneedle for producing the microneedle according to claim 2 or the microneedle array having a plurality of the microneedles wherein the microneedles are arranged side by side, the production method of microneedle comprising:
a needle forming process of producing the needle main body with the flow path blocked with a soluble material dissolvable in a predetermined solution;
a coating process of first masking a part of the surface of the needle main body with a protective member, and subsequently applying the coating member on the surface of the needle main body; and
a flow path forming process of first removing the protective member, and subsequently dissolving the soluble material in the predetermined solution.
